(19) United States
(12) Patent Application Publication
(10) Pub. No.: US 2025/0367432 A1
(43) Pub. Date: Dec. 4, 2025

(54) INTRODUCER SHEATH WITH DUAL ARM HUB WITH SEALS

(71) Applicant: BOSTON SCIENTIFIC SCIMED, INC., Maple Grove, MN (US)

(72) Inventors: Neeraj Kumar Sharma, New Delhi (IN); Qian Liu, Plymouth, MN (US); Anurag Sharma, Gurgaon, Haryana (IN)

(73) Assignee: BOSTON SCIENTIFIC SCIMED, INC., Maple Grove, MN (US)

(21) Appl. No.: 19/221,825

(22) Filed: May 29, 2025

Related U.S. Application Data
(60) Provisional application No. 63/653,452, filed on May 30, 2024.

(51) Int. Cl. A61M 60/865; A61M 60/122; A61M 60/237; A61M 60/419

(52) U.S. Cl. CPC A61M 60/865; A61M 60/122; A61M 60/237; A61M 60/419

(57) ABSTRACT

An introducer sheath may include a valve hub and an elongate shaft extending from the valve hub. The valve hub may include a main port and a side port. The main port may include a first hub portion, a first seal that is disposed within the first hub portion, and a first actuator that is configured to adjust the first seal between an opened position and a closed position. The side port may include a second hub portion, a second seal that is disposed within second hub portion, and a second actuator that is configured to adjust the second seal between an opened position and a closed position.

### CROSS REFERENCE TO RELATED APPLICATIONS

The present application claims the benefit of U.S. Provisional Patent Application Ser. No. 63/653,452, filed May 30, 2024, the disclosure of which is incorporated herein by reference.

### TECHNICAL FIELD

The present disclosure pertains to sheaths for delivering intravascular medical devices. More specifically, the present disclosure relates to a hub of a sheath with a port for securing and/or sealing medical devices within the hub.

### BACKGROUND

In various procedures for delivering intravascular medical devices, a sheath is inserted into a blood vessel of a patient, for example a femoral artery, and one or more medical devices may be advanced through the sheath and into the patient's vasculature. In various instances, the medical devices include catheters, guidewires, or other devices, such as a blood pump. A hub may be incorporated at a proximal end of the sheath to provide access to a lumen of an elongate shaft of the sheath. The hub may include one or more seals, e.g., hemostasis seals, to reduce blood leakage as devices are being inserted, positioned, and removed.

### BRIEF SUMMARY

This disclosure provides design, material, manufacturing method, and use alternatives for medical devices, including introducer and/or repositioning sheaths.

A first example is directed to an introducer sheath that may include a valve hub defining a main port and a side port and an elongate shaft extending from the valve hub, wherein the main port may include a first hub portion, a first seal, the first seal is disposed within the first hub portion, and a first actuator, the first actuator is configured to adjust the first seal between an opened position and a closed position, and wherein the side port may include a second hub portion, a second seal, the second seal disposed within the second hub portion, and a second actuator, the second actuator is configured to adjust the second seal between an opened position and a closed position.

Alternatively or additionally to any of the examples above, the second seal may be biased in an opened position and the second actuator may be configured to engage the second seal to adjust the second seal from the opened position to a closed position.

Alternatively or additionally to any of the examples above, the second seal may be biased closed and the second actuator may be configured to engage the second seal to adjust the second seal from the closed position to the opened position.

Alternatively or additionally to any of the examples above, the second seal may be a compressible seal and the second actuator may comprise a pusher configured to compress and close the second seal.

Alternatively or additionally to any of the examples above, the pusher may include threads configured to engage threads on the second hub portion.

Alternatively or additionally to any of the examples above, the second actuator may comprise a plunger configured to adjust relative to the second hub portion to create an opening through the second seal.

Alternatively or additionally to any of the examples above, the plunger may be configured to extend through the second seal to create the opening through the second seal.

Alternatively or additionally to any of the examples above, the side port may further include a cap configured to couple with the second hub portion and secure the second seal between the cap and the second hub portion, wherein the plunger may be configured to extend around the cap and couple with the second hub portion.

Alternatively or additionally to any of the examples above, the second actuator may be configured to couple with the second hub portion via a snap connection that permits axial movement of the second actuator relative to the second hub portion.

Alternatively or additionally to any of the examples above, the introducer sheath may further include a third seal disposed within the first hub portion.

Alternatively or additionally to any of the examples above, the first actuator may include a pusher, a cap configured to couple with the pusher, and a lock nut configured to engage the cap and the first hub portion to cause the pusher to adjust the first seal from the opened position to the closed position.

Alternatively or additionally to any of the examples above, the second seal may be configured to axially fix an elongate device extending through the second seal when the second seal is in the closed position.

In another example, an introducer sheath may include a valve hub defining a main port and a side port and an elongate shaft extending from the valve hub, wherein the main port may includes a first hub portion, a first seal, the first seal is disposed within the first hub portion, and a second seal, the second seal is disposed within the first hub portion, and the side port may include a second hub portion, a third seal, the third seal is disposed within the second hub portion, and a pusher configured to adjust the second seal between an opened position and a closed position.

Alternatively or additionally to any of the examples above, the third seal may be biased in an opened position and the pusher may be configured to engage the third seal to adjust the third seal from the opened position to a closed position.

Alternatively or additionally to any of the examples above, the pusher may include threads configured to engage threads on the second hub portion as the pusher adjusts the second seal from the opened position to the closed position.

In another example, an introducer sheath may include a valve hub defining a main port and a side port and an elongate shaft extending from the valve hub, wherein the main port may include a first hub portion, a first seal, the first seal is disposed within the first hub portion, and a second seal, the second seal is disposed within the first hub portion, and wherein the side port may include a second hub portion, a third seal, the third seal is disposed within the second hub portion, and a plunger configured to adjust the third seal between an opened position and a closed position.

Alternatively or additionally to any of the examples above, the third seal may be biased closed and the plunger may be configured to engage the third seal to adjust the third seal from the closed position to the opened position.

Alternatively or additionally to any of the examples above, the plunger may be configured to extend through the third seal to create an opening through the third seal.

Alternatively or additionally to any of the examples above, the side port may include a cap configured to couple with the second hub portion and secure the third seal between the cap and the second hub portion, wherein the plunger may be configured to extend around the cap and couple with the second hub portion.

Alternatively or additionally to any of the examples above, the plunger may be configured to couple with the second hub portion via a coupling that permits axial movement of the plunger relative to the second hub portion and limits rotational movement of the plunger relative to the second hub portion.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a schematic side view of an introducer sheath extending into a blood vessel;

FIG. 2 is a schematic cross-section view of a portion of the introducer sheath of FIG. 1 inserted into a blood vessel, and a medical device inserted into the introducer sheath;

FIG. 3 is a schematic perspective view of an illustrative configuration of an introducer sheath;

FIG. 4 is a schematic exploded side view of various components of the introducer sheath of FIG. 3;

FIG. 5 is a schematic exploded perspective view of various components of the introducer sheath of FIG. 3;

FIG. 6 is a schematic cross-section view of a portion of the introducer sheath of FIG. 3;

FIG. 7 is a schematic cross-section view of the introducer sheath of FIG. 3 with medical devices passing through seals of the main and side ports that are in opened positions, with the medical devices schematically depicted in a side view;

FIG. 8 is a schematic cross-section view of the introducer sheath with the medical devices passing through the main and side ports depicted in FIG. 7, with the seals of the main and side ports in closed positions;

FIG. 9 is a schematic exploded side view of various components of an illustrative introducer sheath;

FIG. 10 is a schematic exploded perspective view of various components of the introducer sheath of FIG. 9;

FIG. 11 is a schematic cross-section view of a portion of the introducer sheath of FIG. 9;

FIG. 12 is a schematic cross-section view of the introducer sheath of FIG. 9 with a medical device passing through a main port with a seal in an opened position and a medical device extending into a side port with a seal in a closed position, with the medical devices schematically depicted in a side view;

FIG. 13 is a schematic cross-section view of the introducer sheath with the medical devices passing through the main and side ports depicted in FIG. 12, with the seal of the main port in a closed position, the seal of the side port in an opened position, and the medical device passing through the side port fully inserted;

FIG. 14 is a schematic cross-section view of the introducer sheath with the medical devices passing through the main and side ports depicted in FIG. 13, with the seal of the side port in a closed position;

FIG. 15 is a schematic side view of a portion of an illustrative configuration of an introducer sheath;

FIG. 16 is a schematic cross-section view of a portion of the introducer sheath of FIG. 15, with components of a side port in an exploded view; and

FIG. 17 is a schematic cross-section view of the introducer sheath of FIG. 15, take along line 17-17.

While the disclosure is amenable to various modifications and alternative forms, specifics thereof have been shown by way of example in the drawings and will be described in detail. It should be understood, however, that the intention is not to limit the disclosure to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all embodiments include the particular features, structures, and/or characteristics. Additionally, when particular features, structures, and/or characteristics are described in connection with one configuration, it should be understood that such features, structures, and/or characteristics may also be used connection with other configurations whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure. Additionally, in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

Hemostasis valve hub assemblies of or provided with an introducer sheath may facilitate insertion and positioning of one or more medical devices (e.g., catheters, blood pumps, guidewires, etc.) through the introducer sheath while also helping to prevent blood from leaking during a medical procedure. Some configurations of the present disclosure may include assemblies with components that assist with fixing (e.g., securing or locking axially, rotationally, etc.) a position of one or more medical devices (e.g., a blood pump, catheter, guidewire, etc.) while maintaining a seal around an elongate shaft of the medical device to reduce blood loss through the hub assembly. Some configurations of the disclosed assemblies may provide components that facilitate convenient repositioning of the one or more medical devices relative to the introducer sheath.

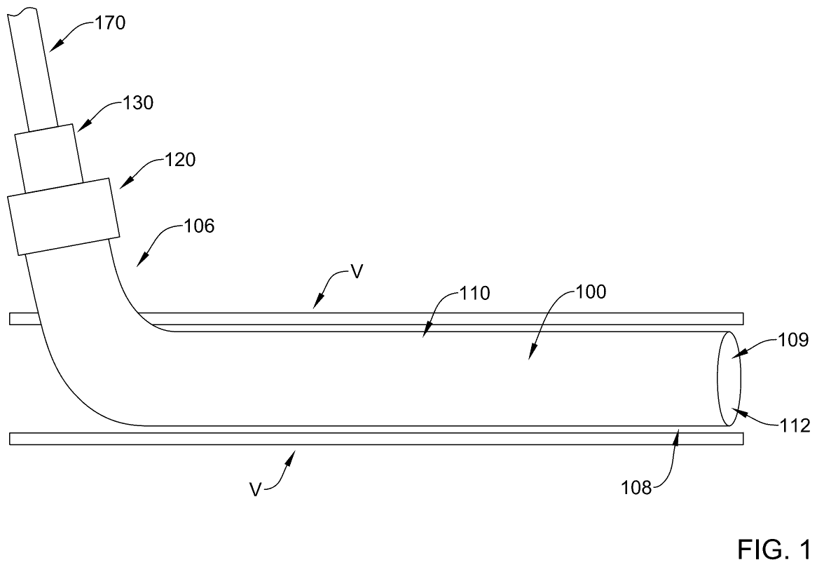

FIG. 1 illustrates a side view of an introducer sheath 100 inserted at least partially into a blood vessel V, shown in cross-section. While the disclosure herein is made with reference largely to the introducer sheath 100, and components thereof, the disclosure may also apply to a repositioning sheath and/or other suitable sheaths.

In some example configurations, the introducer sheath 100 may be used for facilitating the passage of various medical devices, such as catheters, guidewires, blood pumps, and/or other suitable medical devices, through the introducer sheath 100 and into the blood vessel V. In some examples, the introducer sheath 100 may be referred to as a large bore introducer sheath. The introducer sheath 100 may include a proximal end region 106 proximate a proximal end of the introducer sheath 100 and a distal end region 108 proximate a distal end of the introducer sheath 100 that is opposite the proximal end region 106. A body portion 110 of the introducer sheath 100 may extend between the proximal end region 106 and the distal end region 108, and the body portion 110 may entirely or at least partially define a lumen 112 of the introducer sheath 100. The introducer sheath 100 may include a proximal opening (not shown) proximate the proximal end region 106 and a distal opening 109 proximate the distal end region 108, with the lumen 112 extending between the proximal opening and the distal opening 109.

The introducer sheath 100, or components thereof, may be formed by various materials, such as polymeric and/or metallic materials. In some examples, the introducer sheath 100, such as an elongate shaft of the introducer sheath 100, may include a surface coating, such as but not limited to, silicone, PET, or other applicable polymer.

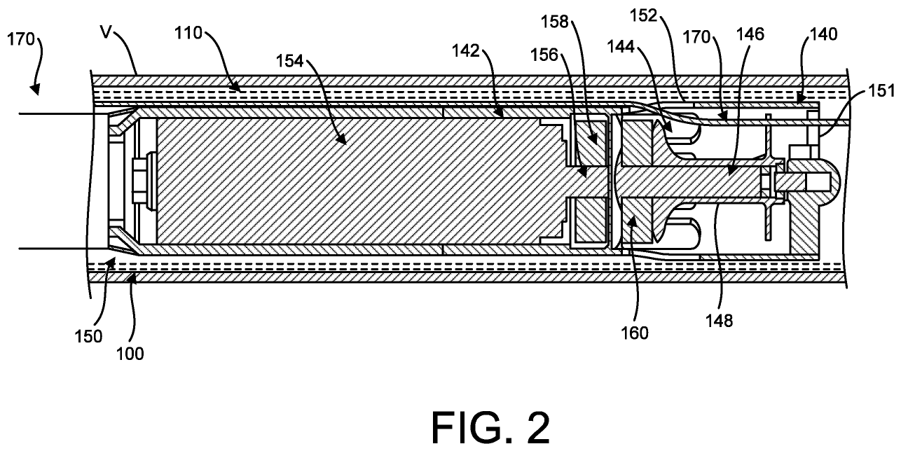

A hemostasis valve hub 120 (hereinafter “hub” for brevity) may be provided at the proximal end region 106 to provide access to the lumen 112 of the introducer sheath 100. The hub 120 may be configured for hemostasis by, for example, helping to prevent blood from leaking out of the introducer sheath 100 during use. For example, a medical device 170, such as a catheter, blood pump, guidewire, etc. may be inserted through the hub 120 and lumen 112 of the introducer sheath 100 and into the blood vessel V, and the hub 120 may maintain hemostasis between the medical device 170, the introducer sheath 100, and the external surroundings. In some examples, the medical device 170 may include and/or be coupled to a blood pump 150 (e.g., as shown in FIG. 2).

After insertion of the medical device 170 into and/or through the hub 120, fixation (e.g., securing or locking) of the axial and/or rotational position of the medical device 170 may be desired to ensure that the medical device 170 (and any device coupled thereto) is maintained in a proper position during use. It may also be desired for the medical personnel to reposition the medical device 170 after insertion. As such, the hub 120 may include one or more tightening ports 130, provided with or attachable thereto, that provides for the fixation (e.g., securing or locking) of the medical device 170 with respect to the hub 120 and blood vessel V. The hub 120 and tightening port(s) 130 may allow for the repositioning of one or more medical device(s) 170 with respect to the hub 120 and the blood vessel V.

FIG. 2 illustrates a cross-section view of the body portion 110 of the introducer sheath 100 of FIG. 1 upon insertion of a medical device, illustratively a blood pump 150, into the introducer sheath 100 (shown in broken lines in FIG. 2). As noted above, the medical device 170 of FIG. 1 may be coupled to or include the blood pump 150, with the medical device 170 extending outside the blood vessel V and the introducer sheath 100. The blood pump 150 may be advanced through the blood vessel V and positioned in or at a target location, such as a target cardiac location (e.g., the left ventricle), via the introducer sheath 100. While the introducer sheath 100 is illustrated in FIG. 2 with the use of the blood pump 150, various other medical devices may be used in conjunction with the introducer sheath 100 and the hemostasis valve hub 120.

The blood pump 150 may include one or more components. In some examples, the blood pump 150 may include an impeller assembly housing 140 and a motor housing 142. The impeller assembly housing 140 and the motor housing 142 may be integrally or monolithically constructed. Alternatively, the impeller assembly housing 140 and the motor housing 142 may be separate components. The impeller assembly housing 140 may carry an impeller assembly 144 therein. The impeller assembly 144 may include an impeller shaft 146 and an impeller 148 that rotate relative to the impeller assembly housing 140 to drive blood through the blood pump 150. In some examples, the impeller shaft 146 and the impeller 148 may be integrally formed, whereas, in other examples the impeller shaft 146 and the impeller 148 may be separate components.

Rotation of the impeller 148 may cause blood to flow from a blood inlet 151 formed on the impeller assembly housing 140, through the impeller assembly housing 140, and out of a blood outlet 152 formed on the impeller assembly housing 140. As shown in FIG. 2, the inlet 151 may be formed on an end portion of the impeller assembly housing 140 and the outlet 152 may be formed on a side portion of the impeller assembly housing 140. In other examples, the inlet 151 and/or the outlet 152 may be formed on other portions of the impeller assembly housing 140. In some examples, the impeller assembly housing 140 may be coupled to a distally extending cannula, and the cannula may receive and deliver blood to the inlet 151.

The motor housing 142 may carry a motor 154, and the motor 154 may be configured to rotatably drive the impeller 148 relative to the impeller assembly housing 140. In some examples, the motor 154 may rotate a drive shaft 156 coupled with or to a driving magnet 158. Rotation of the driving magnet 158 may cause rotation of a driven magnet 160 that may be coupled with and/or part of the impeller assembly 144. More specifically, in configurations incorporating the impeller shaft 146, the impeller shaft 146 and the impeller 148 may be configured to rotate with the driven magnet 160. In other configurations, the motor 154 may be coupled to the impeller assembly 144 via other components.

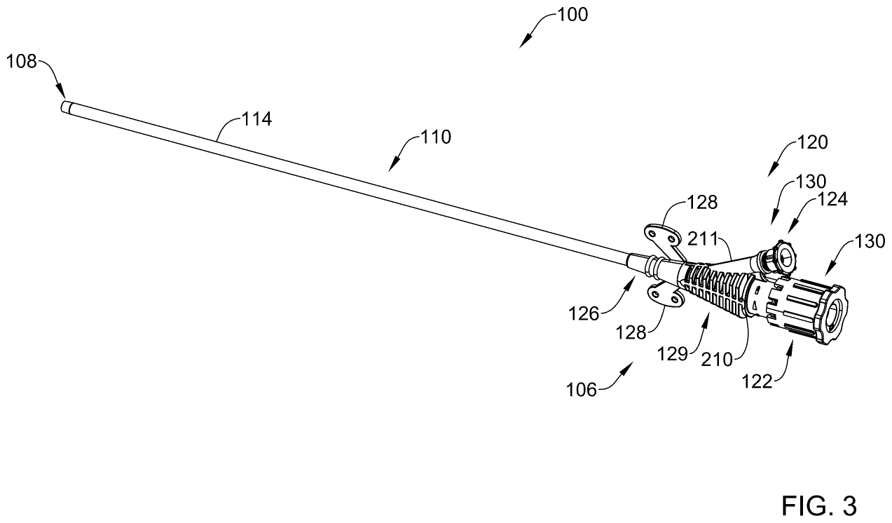

FIG. 3 is a perspective view of an illustrative configuration of the introducer sheath 100. In some examples, the introducer sheath 100 may include the hub 120 and an elongate shaft 114 extending distally from the hub 120 and defining the body portion 110 of the introducer sheath 100.

Although not depicted in FIG. 3, the introducer sheath 100 may include a flush line extending from the hub 120. The flush line may include a tubular member extending from the hub 120 and in fluid communication with a lumen of the hub 120. The tubular member may extend to a stopcock, such as a three-way stopcock, and/or other suitable valve or valve system. The stopcock, when included, may include a first leg coupled to the tubular member, a second leg, a third leg, and a lever that may be rotatable to selectively open/close fluid access between the first, second, and/or third legs. Each of the legs may include a connector, such as a luer connector, as desired.

The hub 120 may include a strain relief 126 configured to provide a flexibility transition along the proximal end region 106. The strain relief 126 may include a body attached to and/or extending from a main body of the hub 120.

The strain relief 126 may include and/or may be coupled with one or more suture pads 128 extending outward therefrom. For example, the strain relief 126 may include first and second suture pads 128 extending from opposite sides of the strain relief 126. The suture pads 128 may facilitate securing the hub 120 against the patient once the introducer sheath 100 has been positioned in the blood vessel of the patient. For example, each suture pad 128 may include at least one opening extending therethrough for receiving a suture used to suture the hub 120 to the skin of the patient.

The suture pads 128 may be angled at an acute angle relative to the longitudinal axis of the introducer sheath 100. For example, the suture pads 128 may be angled toward the distal end region 108 of the introducer sheath 100 such that the free ends of the suture pads 128 are positioned distal of the base ends of the suture pads 128. In some examples, the angle between the suture pads 128 and the longitudinal axis of the introducer sheath 100 may be in a range of about 30 degrees to about 90 degrees, about 35 degrees to about 85 degrees, about 40 degrees to about 80 degrees, about 45 degrees to about 75 degrees, about 50 degree to about 70 degrees, or about 55 degrees to about 65 degrees. In some examples, the angle between the suture pads 128 and the longitudinal axis of the introducer sheath 100 may be about 35 degrees, 40 degrees, 45 degrees, 50 degrees, 55 degrees, or 60 degrees.

A distal end portion of a hub body 129 of the hub 120 may extend into the interior of the strain relief 126 and/or the suture pads 128 and be coupled thereto. For example, the hub body 129 may include an annular rim configured to engage (e.g., form a snap fit) with a mating recess formed in the interior of the strain relief 126 and/or the suture pad(s) 128. In some examples, the suture pad(s) 128 and/or the strain relief 126 may be rotatable relative to the elongate shaft 114 and/or the hub body 129, but other suitable configurations are contemplated.

The hub 120 may include a main port 122, a side port 124, and/or one or more other suitable ports (e.g., one or more suitable ports extending from the main port 122 and/or the side port 124). In some examples, the hub body 129 may include or may be coupled with a hub body portion 210 (e.g., a hub portion) of the main port 122 and/or a hub body portion 211 (e.g., a hub portion) of the side port 124. The main port 122 and/or the side port 124 may provide access to one or more lumens extending through the body portion 110 of the introducer sheath 100. In some examples, the main port 122 and/or the side port 124 may be or may include one or more tightening ports 130.

The side port 124 may extend at any suitable angle relative to the main port 122. In some examples, the side port 124 may extend at an acute angle or a perpendicular angle relative the main port 122. In some examples, the side port 124 may extend at an angle within a range of about twenty degrees to about forty degrees relative to the main port 122. In one example, the side port 124 may extend at an angle of or about twenty-six degrees relative to the main port 122. In one example, the side port 124 may extend at an angle of or about thirty-five degrees relative to the main port 122. In one example, the side port 124 may extend at an angle of or about thirty-eight degrees relative to the main port 122. Other suitable angles between the side port 124 and the main port 122 are contemplated.

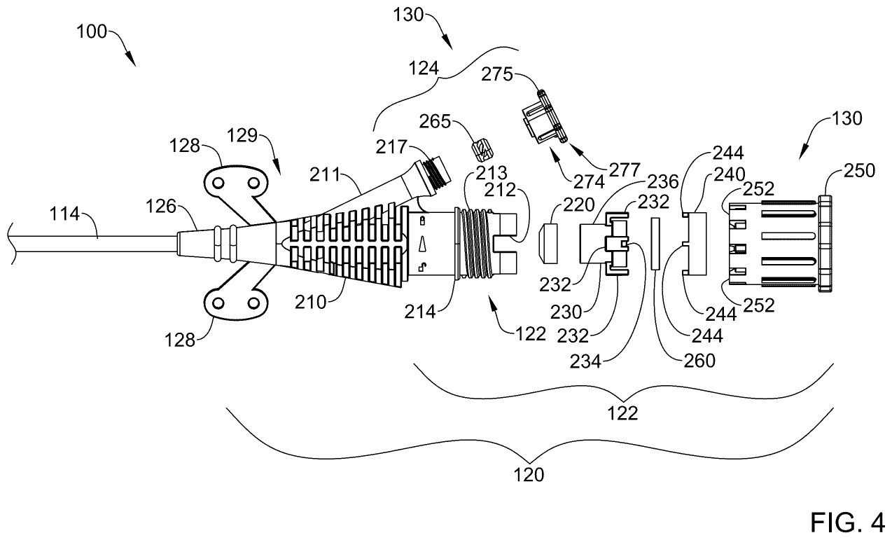

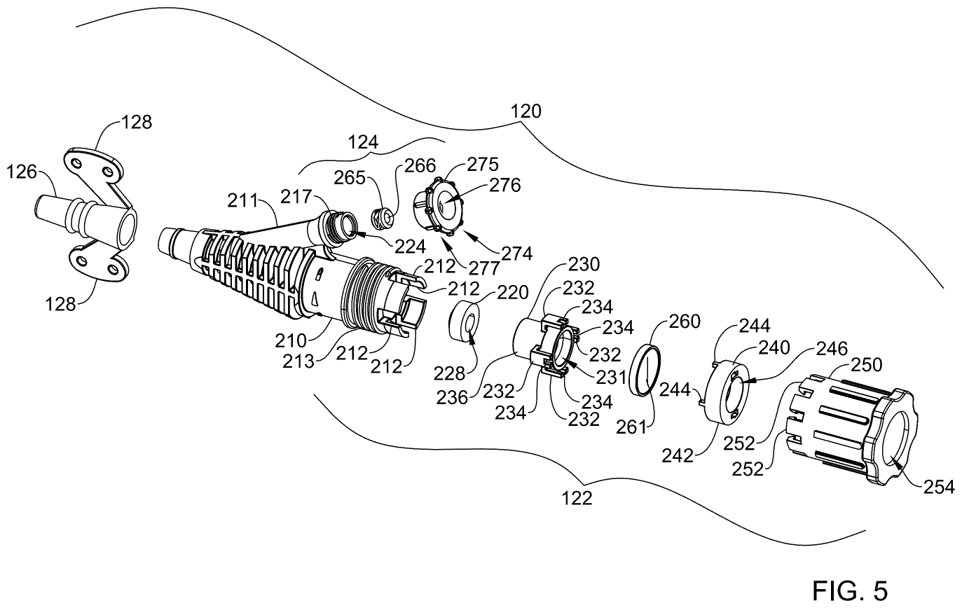

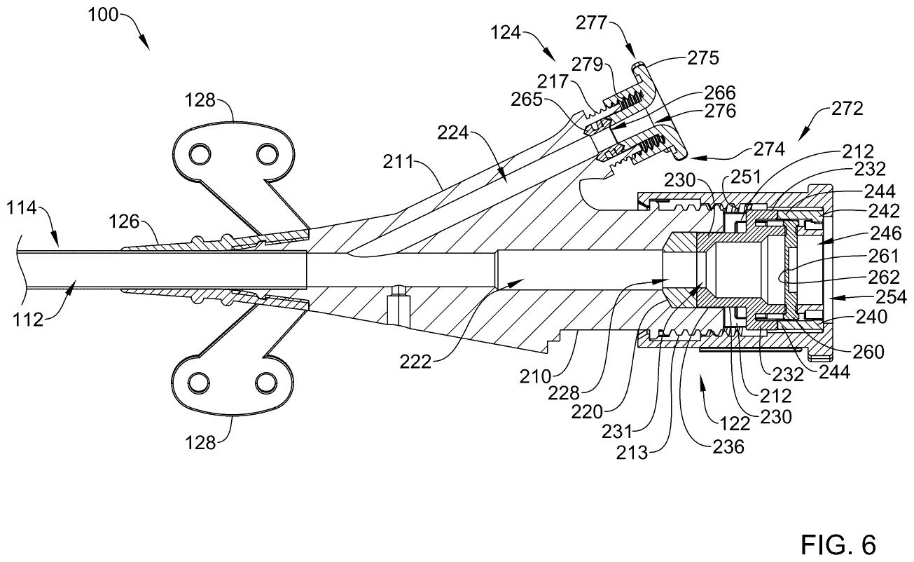

Components of the hub 120 of the illustrative configuration of the introducer sheath 100 depicted in FIG. 3 are described with respect to FIGS. 4-6. FIG. 4 depicts a schematic side view of a portion of the illustrative configuration of the introducer sheath 100 depicted in FIG. 3, with an exploded view of the hub 120. FIG. 5 depicts a schematic perspective exploded view of the hub 120 depicted in FIG. 4. FIG. 6 depicts a schematic cross-section view of the portion of the illustrative introducer sheath 100 depicted in FIG. 4.

The main port 122 of the hub 120 may include the hub body portion 210. In some examples, the hub body portion 210 may be a molded, one-piece structure of the hub body 129. For example, the hub body portion 210 may be molded from a polymeric material and/or other suitable material. In some examples, the hub body portion 210 may be formed of two or more components attached together. In some examples, the hub body portion 210 of the main port 122 may also include or be connected to the hub body portion 211 of the side port 124.

The main port 122 may include a passageway 222 extending therethrough, as depicted for example in FIG. 6. For example, the hub body portion 210 may define the passageway 222. In some examples, the passageway 222 of the main port 122 may converge with a passageway 224 of the side port 124, but other suitable configurations are contemplated. The passageway 222 and/or the passageway 224 may be in fluid communication with the lumen 112 of the introducer sheath 100 extending to the distal opening 109 (See FIG. 1).

The main port 122 may include any suitable number of seals. In some examples, the main port 122 may include a first seal (e.g., a secondary seal) 220 and a second seal (e.g., a primary seal) 260 spaced apart from the first seal 220 along a length of the main port 122. In some examples, the first seal 220 may be configured as a compressible seal (e.g., Tuohy seal) and the second seal 260 may be configured as an elastomeric seal, but other suitable configurations are contemplated. The main port 122, which may be considered a tightening port 130, may further include a pusher 230, a cap 240, a lock nut 250, and/or other suitable components. In some configurations, the pusher 230, the cap 240, and the lock nut 250 may be or operate as an actuator 272 of the main port 122, but other suitable actuator configurations are contemplated for adjusting the first seal 220 between opened and closed states or positions. In operation, rotation of the lock nut 250 may actuate the pusher 230 toward/away from the first seal 220 to adjust the size of an opening 228 through the first seal 220 and open or close the main port 122.

When in the opened state or position, the first seal 220 may allow a medical device to pass through the opening 228. When in the closed state or position, the opening 228 of the first seal 220 may be closed and/or the first seal 220 may seal around a medical device extending through the opening 228 and fix the medical device at an axial and/or rotational position (e.g., axially secure or lock and/or rotationally secure or lock) relative to the hub 120 (e.g., relative to the main port 122 of the hub 120).

The first seal 220 of the main port 122 may be formed from any suitable material. In some examples, the first seal 220 may be formed from material including, but not limited to, elastomeric material, resilient material, compressible material, flexible material, silicone material, polyurethane material, and or other suitable materials. In one example, the first seal 220 may be or may be made from a compressible material to form a compressible seal (e.g., a Tuohy seal), but other suitable configurations are contemplated.

As shown in FIG. 6, the first seal 220 may be positioned in the interior of the hub body portion 210 with the opening 228 of the first seal 220 axially aligned with the passageway 222 of the main port 122 and an opening or lumen 231 of or extending through the pusher 230. A distal end region of the pusher 230 may be positioned in the interior of the hub body portion 210 with a distal end surface of the pusher 230 juxtaposed with a proximal end surface of the first seal 220. In some examples, the pusher 230 may be positioned between the first seal 220 and the second seal 260.

The pusher 230 may have any suitable configuration to facilitate compressing the first seal 220. In some examples, the pusher 230 may include one or more extensions 232 each having one or more slots 234. The extensions 232 may extend radially outward from a body 236 of the pusher 230, but other suitable configurations are contemplated. In some examples, the pusher 230 may include four extensions 232, but other suitable number of extensions 232 are contemplated. In some examples, the extensions 232 may be aligned with one or more slots 212 of the hub body portion 210 of the main port 122, where the extensions 232 may be received within the slots 212. In some examples, the slots 212 may allow limited axial movement of the pusher 230 while restricting rotational movement of the pusher 230. In one example, the slots 212 may allow the pusher 230 to adjust axially a distance of about two millimeters (mm) and/or other suitable distance for adjusting the first seal 220 between the closed position and the opened position.

The second seal 260 of the main port 122 may be located between the pusher 230 and the cap 240, such that the second seal 260 may be captured between a proximal facing end surface of the pusher 230 and a distal facing end surface of the cap 240. The cap 240 may extend around and distally beyond the second seal 260 such that a distal end region of the cap 240 surrounds a proximal end region of the pusher 230 and/or engages the pusher 230, as depicted for example in FIG. 6. Other suitable relative configurations of the pusher 230, the cap 240, and the second seal 260 are contemplated.

The second seal 260 may be any suitable type of seal. For example, the second seal 260 may be an elastomeric seal, a slit valve (e.g., a cross-slit valve), a dome valve, a duckbill valve, and/or any other suitable type of seal or valve configured to seal around an elongate shaft of a medical device when passed therethrough. In one example, the second seal 260 may be a slit valve seal.

The second seal 260 may include one or more slits (e.g., crossing slits) extending entirely through a seal wall and/or one or more slits (e.g., crossing slits) extending only partially through the seal wall. For example, the second seal 260 may be a cross-slit valve having a first slit 261 extending into the wall of the second seal 260 from a first side of the seal but not extending entirely through the wall of the second seal 260 and a second slit 262 extending into the wall of the second seal 260 from a second, opposite side of the second seal 260 but not extending entirely through the wall of the second seal 260. The first slit 261 may intersect the second slit 262 within the wall of the second seal 260. In some examples, the first slit 261 may be arranged perpendicular to the second slit 262, but other suitable configurations are contemplated.

The second seal 260 may be formed from any suitable material. In some examples, the second seal 260 may be formed from material including, but not limited to, elastomeric material, resilient material, compressible material, flexible material, silicone material, polyurethane material, and or other suitable materials. In one example, the second seal 260 may be formed from a resilient, elastomeric material, but other suitable configurations are contemplated.

The cap 240 may have any suitable configuration for engaging or coupling with the pusher 230 and/or the second seal 260. In some examples, the cap 240 may include an opening or lumen 246 extending through a body 242 of the cap 240, one or more feet or tabs 244 extending distally from the body 242 and configured to be positioned in the slots 234 formed in the extension 232 of the pusher 230, and/or one or more other suitable features or components. In one example, the feet or tabs 244 of the cap 240 may be configured to engage the slots 234 to couple the cap 240 with the pusher 230.

The cap 240 may include any suitable number of tabs 244. In some examples, the cap 240 may include four tabs 244 circumferentially spaced (e.g., equally) around the cap 240, but other suitable configurations are contemplated. The tabs 244 may be positioned in four slots 234, respectively, formed in the pusher 230. Positioning the tabs 244 in the slots 234 may prevent or mitigate rotational movement of the cap 240 relative to the pusher 230 and the hub body portion 210 of the main port 122 when the extensions 232 are in the slots 212.

The lock nut 250 may have any suitable configuration. For example, the lock nut 250 may include one or more legs 252, one or more openings 254, threads 251, and/or other suitable features. The lock nut 250 may be configured to couple with the hub body portion 210 of the main port 122 via the legs 252 and/or the threads 251, but the lock nut 250 may include one or more additional or alternative features configured to couple with the hub body portion 210. In some examples, the lock nut 250 may be configured to engage the cap 240 and the hub body portion 210 of the main port 122 to cause the pusher 230 to adjust the first seal 220 from the opened position to the closed position.

The one or more legs 252 of the lock nut 250 may be configured to engage a ridge 214 (e.g., a circumferential ridge and/or other suitable ridge) extending around the hub body portion 210 of the main port 122. In one example, the legs 252 may be configured to snap onto or over the ridge 214 to restrict unintentional separation of the lock nut 250 and the hub body portion 210, while allowing the lock nut 250 to rotate and move axially relative to the hub body portion 210. Alternatively or additionally, the hub body portion 210 may include one or more recesses or indentations configured to receive one or more protrusions from the legs 252. The legs 252 and/or the lock nut 250 may be configured to couple with the hub body portion 210 in one or more other suitable manners.

The threads 251 of the lock nut 250 may be configured to engage threads 213 on the hub body portion 210 of the main port 122 and facilitate advancing the lock nut 250 axially relative to the hub body portion 210 of the main port 122. As depicted in FIGS. 4-6, the hub body portion 210 may include external threads 213 and the lock nut 250 may include internal threads 251, but other suitable configurations are contemplated.

The lock nut 250 may be assembled on the hub body portion 210 such that the lock nut 250 is positioned around all of or at least one of one or more of the first seal 220, the pusher 230, the second seal 260, and the cap 240. When so configured, as discussed herein or otherwise, in operation the threads 251 of the lock nut 250 may engage the threads 213 of the hub body portion 210 as the lock nut 250 is rotated causing the lock nut 250 to advanced axially. When the lock nut 250 is rotated in a first direction and advanced distally, the lock nut 250 may engage a proximal end of the cap 240 to advance the pusher 230 distally, which exerts a compressive force on the first seal 220 to thereby reduce the diameter of the opening 228 through the first seal 220 and adjust the first seal 220 to the closed state or position. The lock nut 250 may be rotated in a second direction opposite of the first direction to advance the lock nut proximally, which may remove or reduce the compressive force on the first seal 220, enlarge the opening 228 through the first seal 220, and adjust the first seal 220 to the opened state or position.

Although the main port 122 has been described with features thereof arranged in a certain axial relationship, other configurations are contemplated. For example, the position of the second seal 260 and the first seal 220 may be reversed, with the second seal 260 located distal of the first seal 220. In some examples, the pusher 230 may be positioned proximal of the lock nut 250 with the pusher 230 located between the lock nut 250 and the first seal 220. In such configurations, as the lock nut 250 is rotated in a first rotational direction relative to the hub body portion 210, the lock nut 250 may urge the pusher 230 to travel proximally relative to the hub body portion 210 to exert a compressive force on the first seal 220 (e.g., located proximal of the pusher 230) to thereby reduce the diameter of the opening 228 through the first seal 220. As the lock nut 250 is rotated in a second rotational direction relative to the hub body portion 210, the lock nut 250 and/or a bias force of the first seal 220 may urge the pusher 230 to travel distally relative to the hub body portion 210 to reduce and/or remove the compressive force on the first seal 220 to increase the diameter of the opening 228 through the first seal 220. Other arrangements are contemplated for positioning the lock nut 250 and/or the pusher 230 relative to the first seal 220 to apply a compressive force thereto.

The openings or lumens 228, 231, 246, 254 of or through components of the main port 122 may be configured to accommodate any suitable size of medical device. In some examples, the main port 122 (e.g., a hemostasis port) may be configured to accommodate a medical device having an outer diameter of 12 F or less, 12 F or more, 14 F or more, 15 F or more, 16 F or more, 17 F or more, and/or other suitable outer diameter sizes. In other words, the first seal 220 and the second seal 260 of the main port 122 may be configured to permit a medical device having an outer diameter of 12 F or less, 12 F or more, 14 F or more, 15 F or more, 16 F or more, 17 F or more, and/or other suitable size, to pass therethrough while maintaining hemostasis.

The side port 124 may include the passageway 224 extending therethrough (see e.g., FIG. 6) and a side port seal 265 positioned along the passageway 224 of the side port 124. In some examples, the hub body portion 211 of the side port 124 may entirely or at least partially define the passageway 224 and the side port seal 265 may be adjusted to open and close the passageway 224. For example, the side port seal 265 may be movable between an open state or position, allowing a medical device to pass through the opening 228, and a closed state, sealing the side port seal 265 around the medical device. In some examples, when the side port seal 265 is in the closed state or position around the medical device, the side port seal 265 may fix the medical device at an axial and/or rotational position (e.g., axially secure or lock and/or rotationally secure or lock) relative to the hub 120 (e.g., relative to the side port 124 of the hub 120).

The side port seal 265 may be formed from any suitable material. In some examples, the side port seal 265 may be formed from material including, but not limited to, elastomeric material, resilient material, compressible material, flexible material, silicone material, polyurethane material, and or other suitable materials. In one example, the side port seal 265 may be or may be made from a compressible material to form a compressible seal (e.g., a Tuohy seal), but other suitable configurations are contemplated.

The side port seal 265 may have any suitable configuration configured to adjust between a closed position and an opened position. In some examples, the side port seal 265 may include a lumen or opening 276 extending therethrough and peaked distal and/or proximal ends (e.g., see FIG. 6). The peaked distal and/or proximal ends may facilitate maintaining a liquid-tight seal between the side port seal 265 and rigid components (e.g., the hub body portion 211 and/or an actuator 274, which may include features configured to mate with and/or receive the peaked ends). Alternatively or additionally, the rigid components may include peaked features configured to engage indentations or other features of the side port seal 265. In some example configurations, the side port seal 265 may include one or more features or designs (e.g., cut-outs, slits, etc.) configured to facilitate the side port seal 265 adjusting between a position to which the side port seal 265 is biased (e.g., an opened position) and an actuated position (e.g., a closed position), but other suitable configurations are contemplated.

In the example configuration of the hub 120 depicted in FIGS. 3-6, the side port 124 may be considered a tightening port 130, which may include the actuator 274. The actuator 274 may include one or more components configured to adjust the side port seal 265 between a closed position and an open position when actuated. As depicted in FIGS. 3-6, the actuator 274 of the side port 124 may be or may include a pusher 275 and/or other suitable components configured to adjust relative to the hub body portion 211 and engage the side port seal 265.

The pusher 275 of the side port 124 may have any suitable configuration configured to adjust the side port seal 265 between the closed position and the opened position. In some examples, the pusher 275 may have a lumen 276 extending therethrough and configured to receive a medical device. In some examples, the pusher 275 may include threads 279 (e.g., internal or external threads) configured to engage threads 217 on the hub body portion 211 of the side port 124. Although other configurations are contemplated, the pusher 275 may include indentations and/or protrusions 277 to facilitate actuating (e.g., rotating) the pusher 275 relative to the hub body portion 211.

In some examples, the threads 279 of the pusher 275 may be configured to engage the threads 217 on the hub body portion 211 of the side port 124 such that when the pusher 275 is rotated the pusher 275 advances axially toward and/or away from the side port seal 265 to adjust a size of the opening 266. In one example, the pusher 275 may be rotated with the threads 279 of the side port seal 265 engaging threads 217 of the hub body portion 211 to advance the pusher 275 toward the side port seal 265 and engage the side port seal 265 such that the side port seal 265 may adjust from an opened position to a closed position. In some examples, the pusher 275 may be configured to close the side port seal 265 (e.g., close the opening 266) such that fluid does not cross the side port seal 265 through the opening 266 when no medical device is passing through the opening and/or when a medical device is passing through the opening 266.

Although the side port 124 has been described with features thereof arranged in a certain axial relationship, other configurations are contemplated. For example, the position of the side port seal 265 and the pusher 275 of the side port 124 may be reversed, with the pusher 275 located distal of the side port seal 265. In such configurations, as the pusher 275 is rotated in a first rotational direction relative to the hub body portion 211, the pusher 275 may travel proximally relative to the hub body portion 211 to exert a compressive force on the side port seal 265 (e.g., located proximal of the pusher 275) to thereby reduce the diameter of the opening 266 through the side port seal 265. As the pusher 275 is rotated in a second rotational direction relative to the hub body portion 211, the pusher 275 and/or a bias force of the side port seal 265 may urge the pusher 275 to travel distally relative to the hub body portion 211 to reduce and/or remove the compressive force on the side port seal 265 and increase the diameter of the opening 266 through the side port seal 265. Other arrangements are contemplated for positioning the pusher 275 relative to the side port seal 265 to apply a compressive force thereto.

The openings or lumens 266, 276 of or through components of the side port 124 may be configured to accommodate any suitable size of medical device. In some examples, the side port 124 may be configured to accommodate a medical device having an outer diameter of 5 F or less, 7 F or less, 9 F or less, 11 F or less, 12 F or less, and/or other suitable sizes of medical devices. In other words, the side port seal 265 of the side port 124 may be configured to permit a medical device having an outer diameter of 5 F or less, 7 F or less, 9 F or less, 11 F or less, 12 F or less, and/or other suitable size, to pass therethrough while maintaining hemostasis.

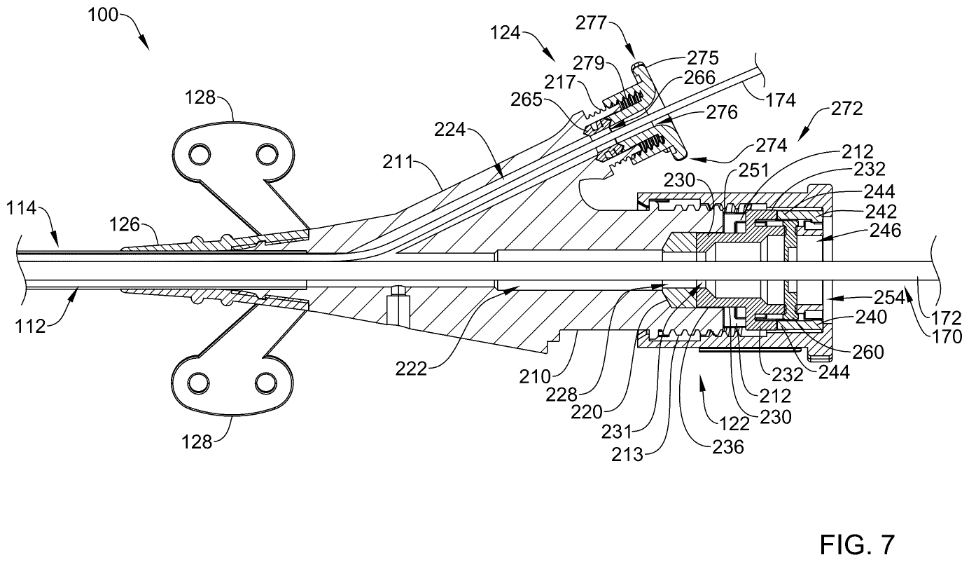

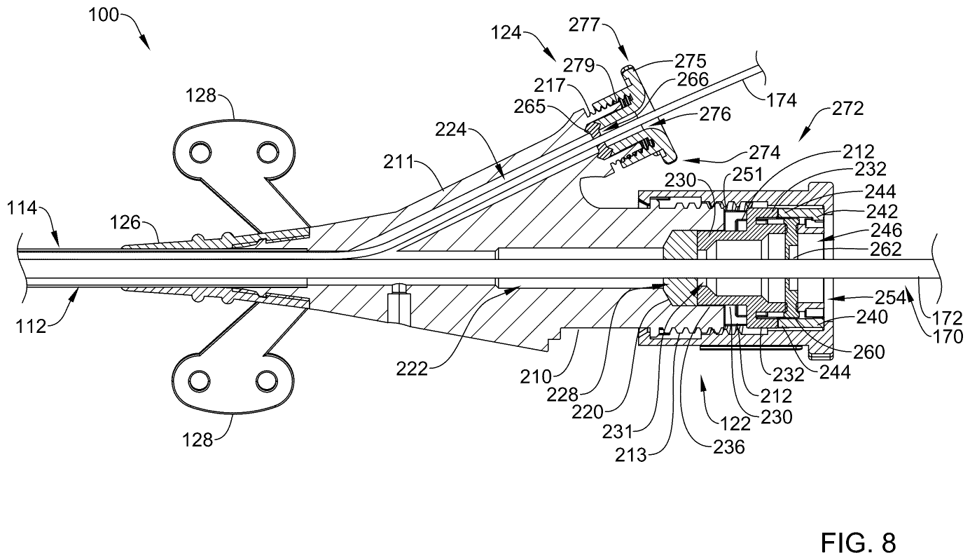

FIGS. 7 and 8 schematically depict positioning and fixing (e.g., axially securing or locking and/or rotationally securing or locking) a first elongate shaft 172 (e.g., a shaft of the medical device 170 and/or other suitable elongate shaft) extending through the main port 122 of the introducer sheath 100 and a second elongate shaft 174 extending through the side port 124 of the introducer sheath 100, where the elongate shafts may be solid shafts and/or may include a lumen (e.g., forming a tube or other suitable structure with a lumen). FIG. 7 is a cross-section view of a portion of the illustrative configuration of the introducer sheath 100 depicted in FIG. 3, with the main port 122 and the side port 124 in the open state or position and the elongate shafts 172, 174 extending therethrough, respectively. FIG. 8 is a cross-section view of a portion of the illustrative configuration of the introducer sheath 100 depicted in FIG. 7, with the main port 122 and the side port 124 in the closed state or position.

As depicted in FIG. 7, the first elongate shaft 172 may be inserted through the second seal 260 of the main port 122, through the lumen 231 of the pusher 230, through the opening 228 of the first seal 220 of the main port 122, and into the passageway 222 of the main port 122. For example, the first elongate shaft 172 may be passed through an opening of the second seal 260 (e.g., through the slit(s) 261, 262) and cause the second seal 260 to flex and/or deform to permit the first elongate shaft 172 to be advanced therethrough. The second seal 260 may seal around an outer perimeter of the first elongate shaft 172 and substantially prevent blood from escaping from the main port 122. Thereafter, the first elongate shaft 172 may pass through the opening 228 of the first seal 220 into the passageway 222 of the main port 122. The first elongate shaft 172 may be further advanced through the elongate shaft 114 of the introducer sheath 100 into the body of the patient for a diagnostic and/or therapeutic procedure.

The second elongate shaft 174 may be inserted through the lumen 276 in the pusher 275 of the side port 124, through the opening 266 of the side port seal 265 of the side port 124, and into the passageway 224 of the side port 124. The second elongate shaft 174 may be further advanced through the elongate shaft 114 of the introducer sheath 100 into the body of the patient for a diagnostic and/or therapeutic procedure. The second elongate shaft 174 may be positioned in the introducer sheath 100 and/or the patient before, during, and/or after the first elongate shaft 172 is positioned in the introducer sheath 100 and/or the patient.

As shown in FIG. 8, once the first elongate shaft 172 and/or the second elongate shaft 174 is positioned at one or more desired locations within the introducer sheath 100 and/or the vasculature of the patient, the first seal 220 of the main port 122 may be compressed and/or tightened around the perimeter of the first elongate shaft 172 to prevent blood from leaking past first elongate shaft 172 and out of the main port 122 and/or lock the first elongate shaft 172 from axial and/or rotational movement relative to the hub 120. For example, as discussed above, the lock nut 250 may be rotated to distally advance the pusher 230, which in turn exerts a compressive force against the first seal 220, reducing the diameter of the opening 228 through the first seal 220, to seal the first seal 220 around the first elongate shaft 172. To allow for readjustment and/or removal of the first elongate shaft 172, the lock nut 250 may be rotated in an opposite direction to proximally advance the pusher 230, remove the compressive force from the first seal 220, and increase the diameter of the opening 228.

Once the first elongate shaft 172 and/or the second elongate shaft 174 is positioned at one or more desired locations within the introducer sheath 100 and/or the vasculature of the patient, the side port seal 265 of the side port 124 may be compressed and/or tightened around the perimeter of the second elongate shaft 174 to prevent blood from leaking past second elongate shaft 174 and out of the side port 124 and/or lock the second elongate shaft 174 from axial and/or rotational movement relative to the hub 120. For example, as discussed above, the pusher 275 may be rotated to distally advance the pusher 275, which in turn exerts a compressive force against the side port seal 265 of the side port 124, reducing the diameter of the opening 266 through the side port seal 265, to seal the side port seal 265 around the second elongate shaft 174. To allow for readjustment and/or removal of the second elongate shaft 174, the pusher 275 may be rotated in an opposite direction to proximally advance the pusher 275, remove the compressive force from the side port seal 265, and increase the diameter of the opening 266.

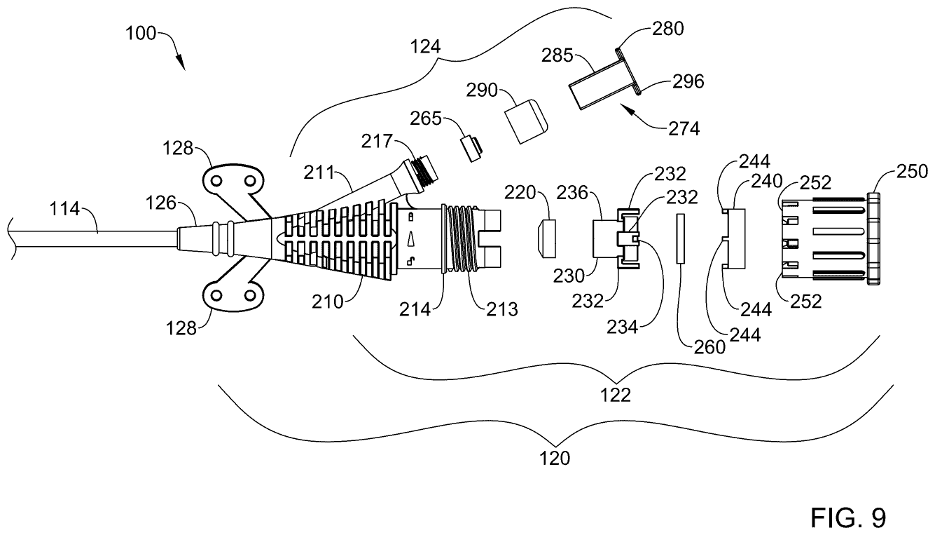

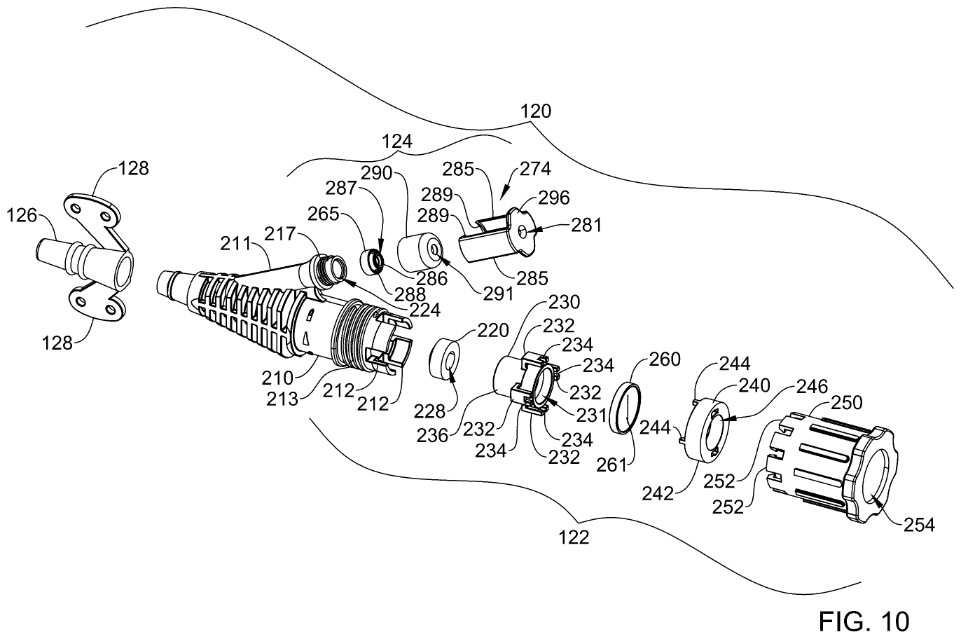

FIGS. 9-11 depict components of the hub 120 of an illustrative configuration of the introducer sheath 100. FIG. 9 depicts a schematic side view of a portion of the illustrative configuration of the introducer sheath 100, with the hub 120 in a schematic exploded view. FIG. 10 depicts a schematic perspective exploded view of the hub 120 depicted in FIG. 9. FIG. 10 depicts a schematic cross-section view of the portion of the illustrative configuration of the introducer sheath 100 depicted in FIG. 9.

The illustrative configuration of the introducer sheath 100 depicted in FIGS. 9-11 may include the hub 120 having the main port 122 and the side port 124, where the main port 122 may be configured identical to or similar to the configuration discussed above with respect to FIGS. 3-8. The configuration of the side port 124 depicted in FIGS. 9-11 may utilize a plunger 280 to adjust the side port seal 265 between the closed state or position and the opened state or position.

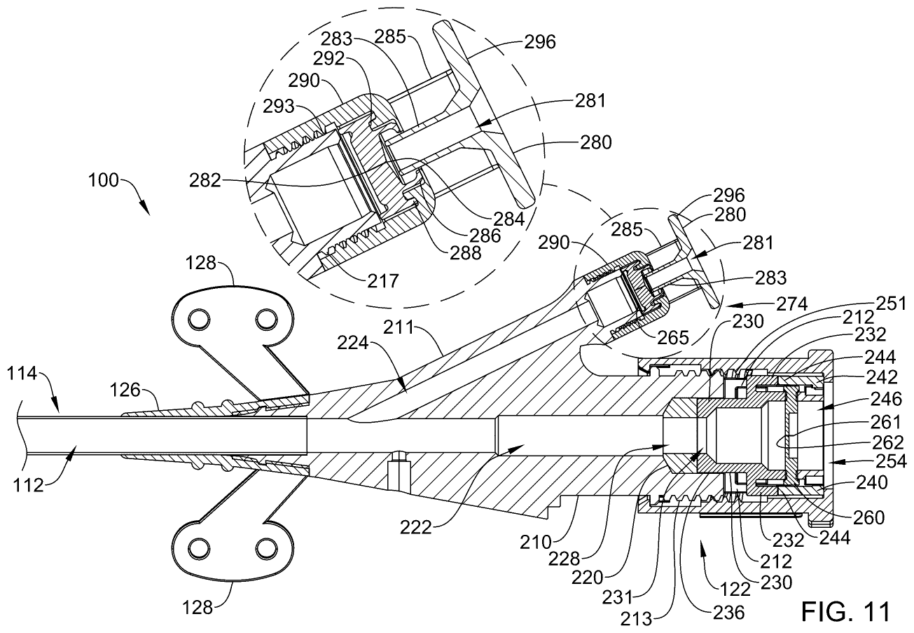

Similar to as discussed herein, the side port 124 may include the passageway 224 extending therethrough and the side port seal 265 positioned along the passageway 224 of the side port 124 (see e.g., FIG. 11). In some examples, the hub body portion 211 of the side port 124 may entirely or at least partially define the passageway 224 and the side port seal 265 may be adjusted to open and close the passageway 224. For example, the side port seal 265 may be movable between an open state or position, allowing a medical device to pass through the opening 228, and a closed state, sealing the side port seal 265 around the medical device. In some examples, when the side port seal 265 is in the closed state or position around the medical device, the side port seal 265 may fix the medical device at an axial and/or rotational position (e.g., axially secure or lock and/or rotationally secure or lock) relative to the hub 120 (e.g., relative to the side port 124 of the hub 120). Although only one side port seal 265 is depicted in FIGS. 9-11, the side port 124 may utilize additional and/or alternatively seals along the passageway 224.

The side port seal 265 may have any suitable configuration configured to adjust between a closed position and an opened position. In some examples, the side port seal 265 may be biased to the closed state or position (e.g., biased closed) and may be configured to adjust to an opened state or position in response to the actuator 274 interacting with the side port seal 265 to form a lumen or opening extending therethrough. In one example configurations, the side port seal 265 may include one or more features or designs (e.g., cut-outs, slits, etc.) configured to facilitate the side port seal 265 adjusting between a position to which the side port seal 265 is biased (e.g., a closed position) and an actuated position (e.g., an opened position), a first ring 286 (e.g., a plunger ring) defining an opening 287 for receiving the plunger 280, and a second ring 288 (e.g., a cap ring) configured to engage a cap 290, but other suitable configurations are contemplated. In some examples, the second ring 288 may be spaced radially outward from the first ring 286 by a gap 292 such that the cap 290 may be received in the gap 292, as depicted for example in FIG. 11. Other suitable configurations of the side port seal 265 are contemplated.

The configuration of the side port seal 265 depicted in FIGS. 9-11 may be any suitable type of seal. For example, the side port seal 265 may be an elastomeric seal, a slit valve (e.g., a cross-slit valve), a dome valve, a duckbill valve, or any other suitable type of seal or valve configured to seal around an elongate shaft of a medical device when passed therethrough. In one example, the side port seal 265 may be a slit valve seal.

The side port seal 265 may include one or more slits (e.g., two or more slits including crossing slits) extending entirely through the seal wall and/or one or more slits (e.g., two or more slits including crossing slits) extending only partially through the seal wall. For example, the configuration of the side port seal 265 depicted in FIGS. 9-11 may be a cross-slit valve having a first slit 282 extending into the wall of the side port seal 265 from a first side of the seal but not extending entirely through the wall of the side port seal 265 and a second slit 284 extending into the wall of the side port seal 265 from a second, opposite side of the side port seal 265 but not extending entirely through the wall of the side port seal 265. The first slit 282 may intersect the second slit 284 within the wall of the side port seal 265, as depicted for example in FIG. 11. In some examples, the first slit 282 may be arranged perpendicular to the second slit 284, but other suitable configurations are contemplated. Other configurations of the side port seal 265 are contemplated.

The side port seal 265 may be formed from any suitable material. In some examples, the side port seal 265 may be formed from material including, but not limited to, elastomeric material, resilient material, compressible material, flexible material, silicone material, polyurethane material, and or other suitable materials. In one example, the side port seal 265 may be or may be made from a resilient, elastomeric material to form a flat seal, but other suitable configurations are contemplated.

The side port seal 265 may be located and/or secured between the hub body portion 211 of the side port 124 and the cap 290. For example, the side port seal 265 may be captured and/or secured between a proximal facing end surface of the hub body portion 211 and a distal facing end surface of the cap 290. In some examples, the cap 290 may extend around and distally beyond the side port seal 265 such that a distal end region of the cap 290 surrounds a proximal end region of the hub body portion 211 and/or engages the hub body portion 211.

The cap 290 may have any suitable configuration configured to couple with the hub body portion 211 and/or a component coupled with the hub body portion 211 to secure the side port seal 265 relative to the hub body portion 211. In one example, the cap 290 may be configured to couple with the hub body portion 211 via threads 293 on the cap 290 and threads 217 on the hub body portion 211, as depicted for example in FIG. 11, but the cap 290 may be coupled with the hub body portion 211 in one or more additional or alternative manners that facilitates securing the side port seal 265 relative to the hub body portion 211. In some examples, the cap 290 may be omitted if the side port seal 265 is secured to or relative to the hub body portion 211 in one or more other suitable manners.

The cap 290 may include a seal ring 294, which may be an annular indentation portion configured to receive the cap ring 288 of the side port seal 265. When the cap 290 is coupled with the hub body portion 211 of the side port 124, the seal ring 294 may receive the cap ring 288 of the side port seal 265, as depicted for example in FIG. 11. Other suitable relative configurations of the cap 290 and the side port seal 265 are contemplated.

In the example configuration of the hub 120 depicted in FIGS. 9-11, the side port 124 may include one or more actuators 274 configured to adjust the side port seal 265 between a closed position and an open position when actuated. For example, an illustrative configuration of the actuator 274 of the side port 124 may be or may include the plunger 280 and/or other suitable components configured to adjust relative to the hub body portion 211, the cap 290, and/or the side port seal 265 to engage and/or adjust the side port seal 265 between the closed state or position and an opened state or position.

The plunger 280 of the side port 124 may have any suitable configuration configured to adjust the side port seal 265 between the closed position and the opened position. In some examples, the plunger 280 may have a lumen 281 extending therethrough and configured to receive a medical device. In some examples, the plunger 280 may include one or more legs 285 configured to extend around the cap 290 and engage or otherwise couple with the hub body portion 211 of the side port 124. Although two legs 285 are depicted in FIG. 10, other suitable number of legs 285 may be utilized.

In some examples, the legs may include one or more feet 289 configured to engage the hub body portion 211 and/or other suitable component of the side port 124 distal of the threads 217 on the hub body portion 211 to couple the plunger 280 with the hub body portion 211. In some examples, the legs 285 of the plunger 280 may be flexible such that the legs 285 may facilitate coupling the plunger 280 with the hub body portion 211 or other suitable component coupled to the hub body portion 211 with a snap connection between the feet 289 and the hub body portion 211 and/or other suitable component. The legs 285 and the feet 289 of the plunger 280, when included and coupled with the hub body portion 211, may be configured to facilitate axial movement of the plunger 280 relative to side port seal 265, while preventing unintentional separation of the plunger 280 and the hub body portion 211. In one example, the legs 285 and the feet 289 may be configured to engage the hub body portion 211 so as to facilitate about 3 mm of movement of the plunger 280 in the axial direction, but other suitable axial movement distances are contemplated. Alternatively or additionally, the plunger 280 may be configured to engage the hub body portion 211 and/or the cap 290 in one or more other suitable manners including, but not limited to, via threads, keyed components, and/or other suitable connecting or coupling components that may facilitate axial adjustment of the plunger 280.

The plunger 280 may include a stem 283 configured to engage the side port seal 265, as depicted for example in FIG. 11. In some examples, the stem 283 may be elongated along a central axis of the plunger 280 and may be located radially inward from the legs 285. The stem 283 may define the lumen 281 configured to receive a medical device.

The stem 283 may have any suitable configuration for engaging the side port seal 265 and adjusting the side port seal 265 between the closed position and the opened position. In operation, the stem 283 may be configured such that when the plunger 280 is advanced axially toward the side port seal 265, the stem 283 may extend through the side port seal 265 and the lumen 281 through the stem 283 may define an opening of the side port seal 265 and when the plunger 280 is advanced axially away from and out of the side port seal 265, the side port seal 265 may be sealed due to the bias forces thereof and/or may seal around a medical device extending therethrough. In some examples, the stem 283 may be have a length suitable for extending an entire thickness of the side port seal 265 in the axial direction.

In one example, the stem 283 may have a length suitable for extending entirely through the side port seal 265 when the plunger 280 is actuated in a first direction (e.g., where the side port seal 265 may have thickness of about 2 mm and/or other suitable thickness) and for being completely removed from insertion in the side port seal 265 when the plunger 280 is actuated in a second direction opposite of the first direction. The stem 283 may have an inner diameter or other dimension suitable for receiving a desired medical device and an outer diameter configured to extend through the opening 291 in the cap 290 and/or through an opening in the side port seal 265 caused by engagement of the side port seal 265 with the stem 283. In some examples, the outer diameter of the stem 283 may be sized to engage a portion of the cap 290 defining the opening 291 such that the engagement between the stem 283 and the cap 290 acts as a barrier to fluid. The stem 283 may have any suitable shape including, but limited to a cylindrical shape, a rectangular cross-section shape, an oval cross-section shape, a star cross-section shape, and/or other suitable shapes.

The plunger 280 may include a force receiving component 296 configured to receive a force to adjust the plunger axially. In some examples, the force receiving component 296 may include or define a lip having opposing surfaces, where a first surface of the opposing surfaces may be configured to receive a force to advance the plunger 280 in a first axial direction and a second surface of the opposing surfaces may be configured to receive a force to advance the plunger 280 in a second axial direction that is opposite the first direction. In one example, the first surface of the force receiving component 296 may define or at least partially define an opening for the lumen 281 of the plunger 280.

Although the side port 124 has been described with features thereof arranged in a certain axial relationship, other configurations are contemplated. For example, the position of the side port seal 265 and the plunger 280 of the side port 124 may be reversed, with the plunger 280 located distal of the side port seal 265. In such configurations, as the plunger 280 may be advanced proximally relative to the hub body portion 211, the stem 283 may travel proximally relative to the hub body portion 211 to engage the side port seal 265 and extend therethrough to create the opening through the side port seal 265 for receiving a medical device. As the plunger 280 is advanced in a distal direction relative to the hub body portion 211, the plunger 280 and/or a bias force of the side port seal 265 may urge the plunger 280 to travel distally relative to the hub body portion 211 to remove the stem 283 from the side port seal and close the side port seal. Other arrangements are contemplated for positioning the plunger 280 and/or the cap 290 relative to the side port seal 265.

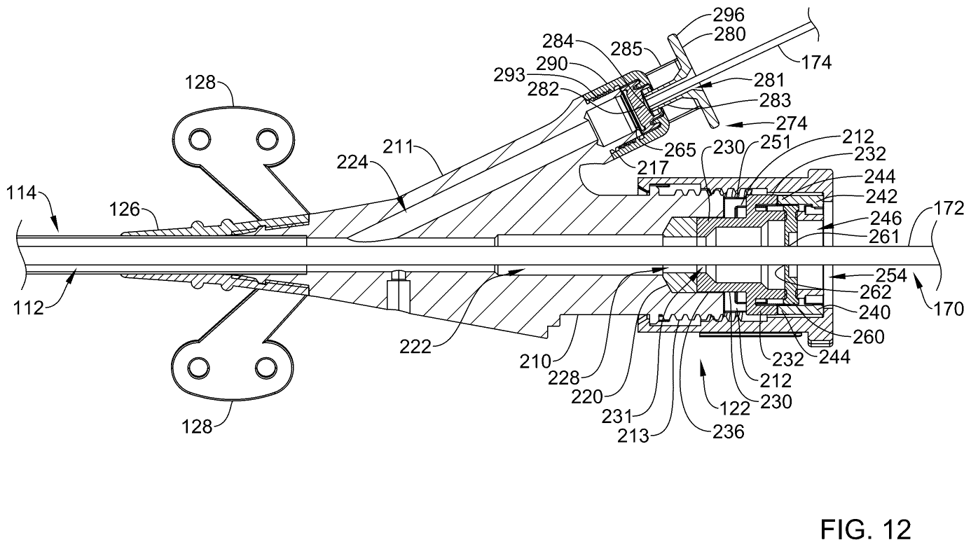

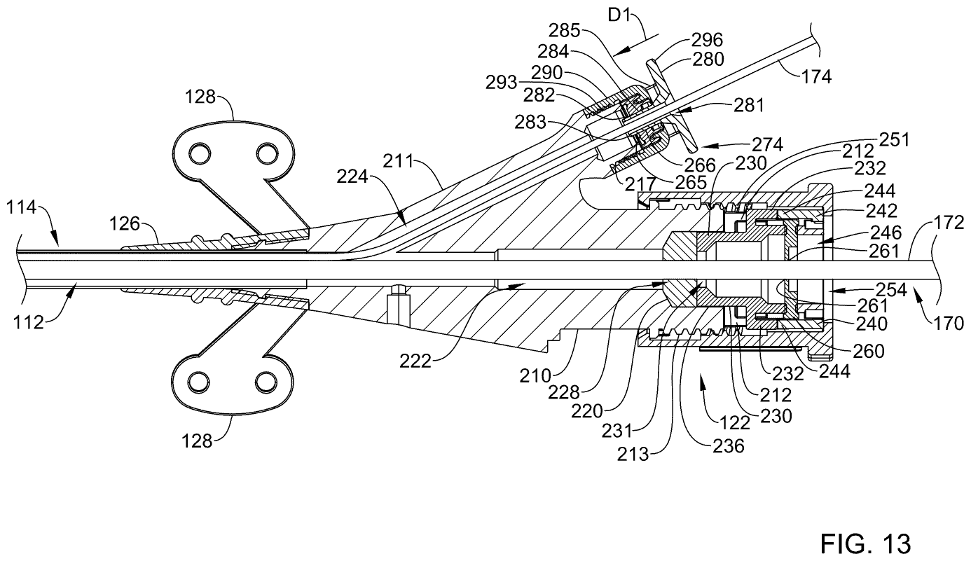

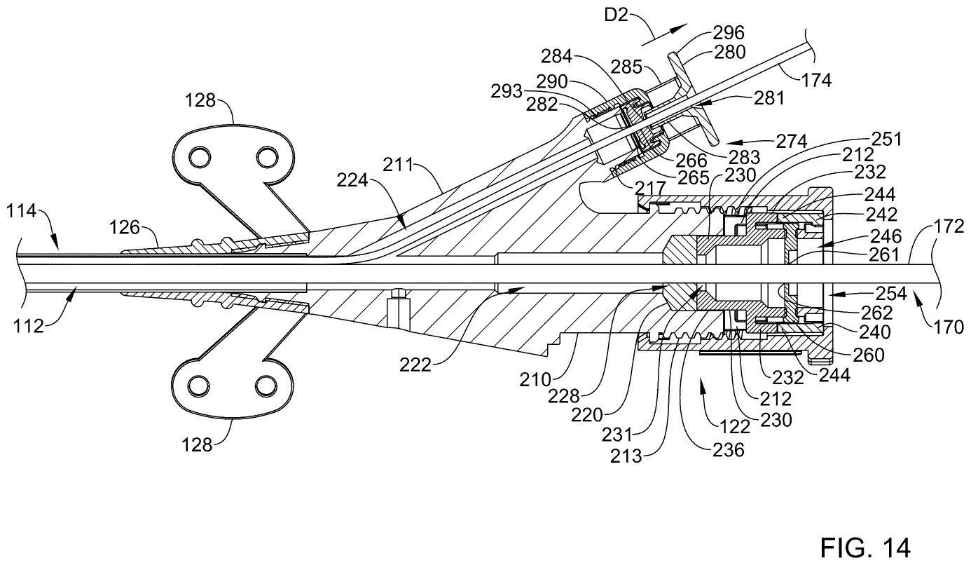

FIGS. 12-14 schematically depict positioning and fixing (e.g., axially securing or locking and/or rotationally securing or locking) a first elongate shaft 172 (e.g., a shaft of the medical device 170 and/or other suitable elongate shaft) extending through the main port 122 of the introducer sheath 100 and a second elongate shaft 174 (e.g., that is a medical device or is part of a medical device) extending through the side port 124 of the introducer sheath 100, where the elongate shafts may be solid shafts and/or may include a lumen (e.g., forming a tube or other suitable structure with a lumen). FIG. 12 is a schematic cross-section view of a portion of the illustrative configuration of the introducer sheath 100 depicted in FIGS. 9-11, with the main port 122 in an open state or position with the first elongate shaft extending therethrough and the side port 124 in the closed state or position with the second elongate shaft 174 extending through the plunger 280, positioned in a first position, to the side port seal 265. FIG. 13 is a schematic cross-section view of a portion of the illustrative configuration of the introducer sheath 100 depicted in FIG. 12, with the main port 122 in the closed state or position and the first elongate shaft 172 extending therethrough and with the side port 124 in the opened state or position and the second elongate shaft 174 extending therethrough. FIG. 14 is a schematic cross-section view of the portion of the illustrative configuration of the introducer sheath 100 depicted in FIG. 13, with the side port 124 in the closed state or position and the second elongate shaft 174 extending therethrough.

As depicted in FIG. 12 and similar to or the same as discussed with respect to FIG. 8, the first elongate shaft 172 may be inserted through the second seal 260 of the main port 122, through the lumen 231 of the pusher 230, through the opening 228 of the first seal 220 of the main port 122, and into the passageway 222 of the main port 122. For example, the first elongate shaft 172 may be passed through an opening of the second seal 260 (e.g., through the slit(s) 261, 262) and cause the second seal 260 to flex and/or deform to permit the first elongate shaft 172 to be advanced therethrough. The second seal 260 may seal around an outer perimeter of the first elongate shaft 172 and substantially prevent blood from escaping from the main port 122. Thereafter, the first elongate shaft 172 may pass through the opening 228 of the first seal 220 into the passageway 222 of the main port 122. The first elongate shaft 172 may be further advanced through the elongate shaft 114 of the introducer sheath 100 into the body of the patient for a diagnostic and/or therapeutic procedure.

The second elongate shaft 174 may be inserted through the lumen 281 in the plunger 280 of the side port 124. In some examples, the second elongate shaft 174 may be advanced through the lumen 281 of the plunger 280 until a distal end reaches a proximal facing surface of the side port seal 265. The second elongate shaft 174 may be positioned in the plunger 280 before, during, and/or after the first elongate shaft 172 is positioned in the introducer sheath 100 and/or the patient. Additionally or alternatively, the second elongate shaft 174 may be first inserted into the plunger 280 after the plunger 280 has been actuated to create an opening through the side port seal 265.

As depicted in FIG. 13, once the first elongate shaft 172 is positioned at one or more desired locations within the introducer sheath 100 and/or the vasculature of the patient, the first seal 220 of the main port 122 may be compressed and/or tightened around the perimeter of the first elongate shaft 172 to prevent blood from leaking past the first elongate shaft 172 and out of the main port 122 and/or lock the first elongate shaft 172 from axial and/or rotational movement relative to the hub 120. For example, as discussed above, the lock nut 250 may be rotated to distally advance the pusher 230, which in turn exerts a compressive force against the first seal 220, reducing the diameter of the opening 228 through the first seal 220, to seal the first seal 220 around the first elongate shaft 172.

To adjust the side port seal 265 to the opened state or position, the plunger 280 may be advanced in a first direction D1 (e.g., in a distal direction, as depicted in FIG. 13) until the stem 283 extends through an entirety of the side port seal 265. In some examples, a force in the first direction D1 may be applied to the force receiving component 296 (e.g., to a proximal facing side or other suitable side of the force receiving component 296) to advance the stem 283 of the plunger 280 distally through the side port seal 265, such that the second elongate shaft 174 may be inserted through lumen 281 of the plunger 280, through the side port seal 265, into the passageway 222 of the main port 122, and through the elongate shaft 114 of the introducer sheath 100 into the body of the patient for a diagnostic and/or therapeutic procedure.

Once the second elongate shaft 174 is positioned at one or more desired locations within the introducer sheath 100 and/or the vasculature of the patient, the plunger 280 may be advanced in a second direction D2 (e.g., in a proximal direction, as depicted in FIG. 14) until the stem 283 is removed (e.g., completely or at least partially removed) from the side port seal 265. In some examples, a force in the direction D2 may be applied to the force receiving component 296 (e.g., a distal facing side or other suitable side of the force receiving component 296) to advance the stem 283 of the plunger 280 proximally out of the side port seal 265, such that the side port seal 265 may close around the second elongate shaft and create a fluid-tight barrier around the second elongate shaft 174, as depicted in FIG. 14. In some examples, a bias force of the side port seal 265 may act on the plunger 280 to move the plunger 280 in the second direction D2 and out of the side port seal 265. The bias force of the side port seal 265 (e.g., a force biasing the side port seal 265 to the closed state or position) may result in the side port seal 265 being compressed around a perimeter of the second elongate shaft 174 to prevent blood from leaking past the side port seal 265 and out of the side port 124 and/or lock the second elongate shaft 174 from axial and/or rotational movement relative to the hub 120.

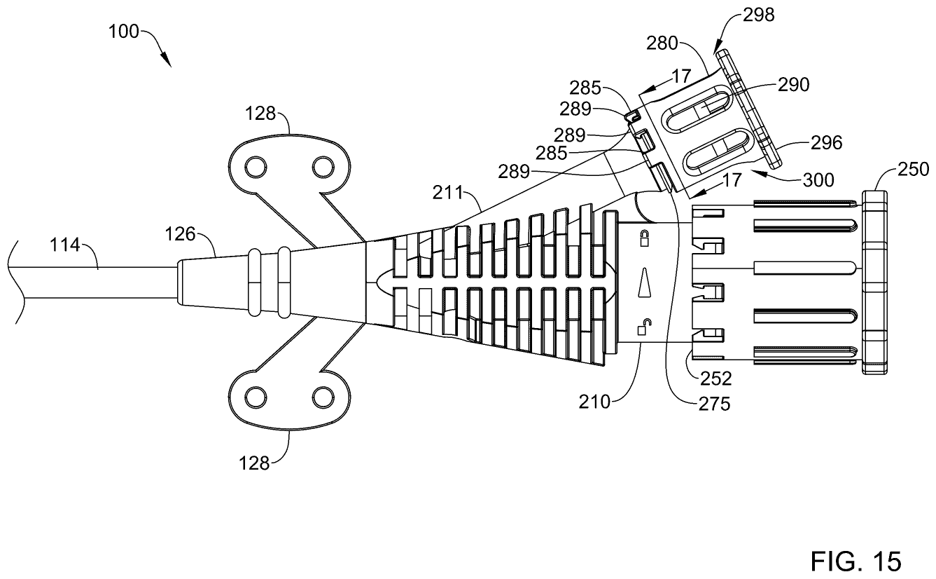

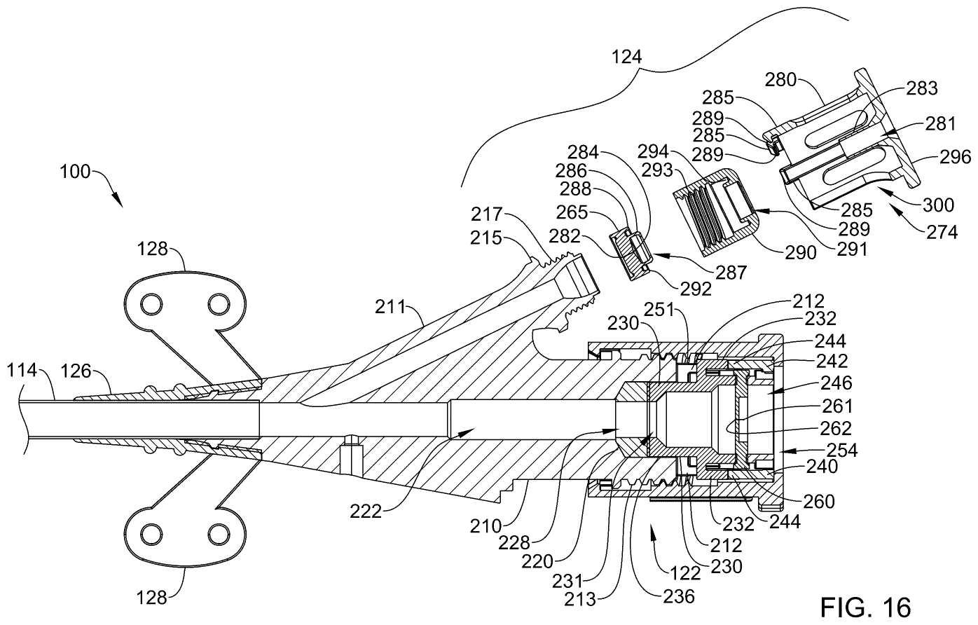

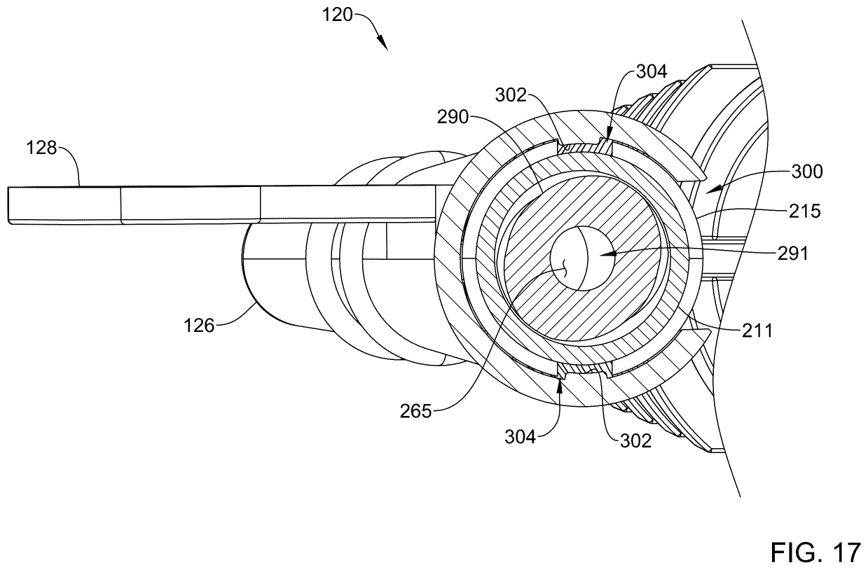

FIGS. 15-17 schematically depict components of the hub 120 of an illustrative configuration of the introducer sheath 100. FIG. 15 depicts a schematic side view of the hub 120 of the illustrative configuration of the introducer sheath 100. FIG. 16 depicts a schematic cross-section view of the hub 120 depicted in FIG. 15, with the components of the side port 124 in an exploded configuration. FIG. 17 depicts a schematic cross-section view of the side port 124 of the hub 120 of the illustrative introducer sheath 100 depicted in FIG. 15, taken along line 17-17.

The illustrative configuration of the introducer sheath 100 depicted in FIGS. 15-17 may include the hub 120 having the main port 122 and the side port 124, where the main port 122 may be configured identical to or similar to the configuration discussed above with respect to FIGS. 3-8. The configuration of the side port 124 depicted in FIGS. 15-17 may be configured similar to the side port 124 discussed with respect to FIGS. 9-14, but with a modified configuration of the plunger 280 and the hub body portion 211 of the side port 124. The configuration of the plunger 280 depicted in FIGS. 15-17 may interact with the side port seal 265 in a similar manner to as discussed with respect to FIGS. 9-14, but may include a body 298 extending between the force receiving component 296 and the legs 285.

The body 298 may have any suitable configuration. In one example, the body 298 may have a cut-out portion 300, where the cut-out portion 300 may face and/or may be proximate the main port 122 and/or the lock nut 250 of the main port 122 such that when both of the plunger 280 and the lock nut 250 are advanced distally, the plunger 280 and lock nut 250 do not engage one another. Alternatively or additionally, the lock nut 250 may include the cut-out portion or the plunger 280 and/or the lock nut 250 may be otherwise configured to advance relative to one another while maintaining a desired angle between the main port 122 and the side port 124.

In some examples, the plunger 280 may include one or more legs 285 with feet 289 configured to engage a ridge 215 of the hub body portion 211 to create a snap connection and/or other suitable connection between the plunger 280 and the hub body portion 211. The connection or coupling between the plunger 280 and the hub body portion 211 or a component coupled with the hub body portion 211 may facilitate advancing the plunger 280 in opposing directions such that the stem 283 of the plunger 280 may create an opening through an entirety of the side port seal 265 and allow for the side port seal 265 to close by removing (e.g., entirely or at least partially) the stem 283 from the side port seal 265 and allowing the bias forces of the side port seal 265 to close around a medical device inserted therethrough and/or otherwise close entirely on itself.

FIG. 17 depicts a keyed connection or coupling between the plunger 280 and the hub body portion 211 of the side port 124. The keyed connection, however, may be between the plunger 280 one or more other suitable components of the side port 124. In some examples, the keyed connection or coupling between the plunger 280 and the hub body portion 211 may include one or more tabs on one or both of the plunger 280 and the hub body portion 211 and one or more recesses of one or both of the plunger 280 and the hub body portion 211 that are aligned with the one or more tabs. The recesses may be configured (e.g., sized, shaped, etc.) in any suitable manner to receive the tabs and may extend partially through or entirely through the ridge 215 or other suitable component of the side port 124. As depicted in FIG. 17, the plunger 280 may include two tabs 302 equally circumferentially spaced from one another that are each configured to mate with one of at least two recess 304 in the ridge 215 of the hub body portion 211 (e.g., where the recesses 304 extend entirely through the ridge 215), where two of the recesses 304 are equally circumferentially spaced from one another and align with the tabs 302. In some examples, engagement of the tabs 302 with the recesses 304 may allow for axial movement of the plunger 280 to adjust the side port seal 265 between the closed state or position and the opened state or position, while limiting or preventing rotational movement of the plunger 280 relative to the hub body portion 211. The limiting or preventing rotational movement of the plunger 280 may facilitate maintaining the cut-out portion 300 of the plunger 280 in a desired position facing or proximate to the main port 122 and the lock nut 250 thereof. Other suitable configurations of the plunger 280 and/or other components of the side port 124 are contemplated.

This disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An introducer sheath, comprising:
a valve hub defining a main port and a side port;
an elongate shaft extending from the valve hub,
wherein the main port includes:
a first hub portion;
a first seal, the first seal is disposed within the first hub portion; and
a first actuator, the first actuator is configured to adjust the first seal between an opened position and a closed position, and
wherein the side port includes:
a second hub portion;
a second seal, the second seal disposed within the second hub portion; and
a second actuator, the second actuator is configured to adjust the second seal between an opened position and a closed position.

2. The introducer sheath of claim 1, wherein the second seal is biased in an opened position and the second actuator is configured to engage the second seal to adjust the second seal from the opened position to a closed position.

3. The introducer sheath of claim 1, wherein the second seal is biased closed and the second actuator is configured to engage the second seal to adjust the second seal from the closed position to the opened position.

4. The introducer sheath of claim 1, wherein the second seal is a compressible seal and the second actuator comprises a pusher configured to compress and close the second seal.

5. The introducer sheath of claim 4, wherein the pusher includes threads configured to engage threads on the second hub portion.

6. The introducer sheath of claim 1, wherein the second actuator comprises a plunger configured to adjust relative to the second hub portion to create an opening through the second seal.

7. The introducer sheath of claim 6, wherein the plunger is configured to extend through the second seal to create the opening through the second seal.

8. The introducer sheath of claim 6, wherein the side port further includes:
a cap configured to couple with the second hub portion and secure the second seal between the cap and the second hub portion; and
wherein the plunger is configured to extend around the cap and couple with the second hub portion.

9. The introducer sheath of claim 1, wherein the second actuator is configured to couple with the second hub portion via a snap connection that permits axial movement of the second actuator relative to the second hub portion.

10. The introducer sheath of claim 1, further comprising:
a third seal disposed within the first hub portion.

11. The introducer sheath of claim 1, wherein the first actuator comprises:
a pusher;
a cap configured to couple with the pusher; and
a lock nut configured to engage the cap and the first hub portion to cause the pusher to adjust the first seal from the opened position to the closed position.

12. The introducer sheath of claim 1, wherein the second seal is configured to axially fix an elongate device extending through the second seal when the second seal is in the closed position.

13. An introducer sheath, comprising:
a valve hub defining a main port and a side port;
an elongate shaft extending from the valve hub,
wherein the main port includes:
a first hub portion;
a first seal, the first seal is disposed within the first hub portion; and
a second seal, the second seal is disposed within the first hub portion, and
wherein the side port includes:
a second hub portion;
a third seal, the third seal is disposed within the second hub portion; and
a pusher configured to adjust the second seal between an opened position and a closed position.

14. The introducer sheath of claim 13, wherein the third seal is biased in an opened position and the pusher is configured to engage the third seal to adjust the third seal from the opened position to a closed position.

15. The introducer sheath of claim 13, wherein the pusher includes threads configured to engage threads on the second hub portion as the pusher adjusts the second seal from the opened position to the closed position.

16. An introducer sheath, comprising:
a valve hub defining a main port and a side port;
an elongate shaft extending from the valve hub,
wherein the main port includes:
a first hub portion;
a first seal, the first seal is disposed within the first hub portion; and
a second seal, the second seal is disposed within the first hub portion, and
wherein the side port includes:
a second hub portion;
a third seal, the third seal is disposed within the second hub portion; and
a plunger configured to adjust the third seal between an opened position and a closed position.

17. The introducer sheath of claim 16, wherein the third seal is biased closed and the plunger is configured to engage the third seal to adjust the third seal from the closed position to the opened position.

18. The introducer sheath of claim 16, wherein the plunger is configured to extend through the third seal to create an opening through the third seal.

19. The introducer sheath of claim 16, wherein the side port further includes:
a cap configured to couple with the second hub portion and secure the third seal between the cap and the second hub portion; and
wherein the plunger is configured to extend around the cap and couple with the second hub portion.

20. The introducer sheath of claim 16, wherein the plunger is configured to couple with the second hub portion via a coupling that permits axial movement of the plunger relative to the second hub portion and limits rotational movement of the plunger relative to the second hub portion.
